(19) United States
(12) Patent Application Publication
(10) Pub. No.: US 2026/0341533 A1
(43) Pub. Date: Oct. 29, 2026

(54) COMPUTER-IMPLEMENTED PREDICTIVE OUTCOME GENERATION AND PATIENT MONITORING COMPUTER SYSTEM THEREOF

(71) Applicant: SOPHiA GENETICS S.A., Rolle (CH)

(72) Inventors: Vivien Pianet, Gradignan (FR); Gilbert Perrin, Préverenges (CH)

(73) Assignee: SOPHiA GENETICS S.A., Rolle (CH)

(21) Appl. No.: 19/109,911

(22) PCT Filed: Sep. 8, 2023
(86) PCT No.: PCT/EP2023/074712

§ 371 Date: Mar. 7, 2025

Related U.S. Application Data
(60) Provisional application No. 63/404,949, filed on Sep. 8, 2022.

(51) Int. Cl. G16H 50/70; G16H 10/60; G16H 50/20

(52) U.S. Cl. CPC G16H 50/70; G16H 10/60; G16H 50/20

(57) ABSTRACT

Clinical decisions are now based on large amounts of data from diverse sources and modalities. The medical personnel (MP) or user is now facing the challenge of leveraging available data to ensure clinical and patient management decisions are based on the most relevant and up-to-date data for the condition and are adapted to the patient profile. One aim of the present invention is to provide the medical personnel or a user with a system for monitoring patient clinical outcome and for supporting clinical decision in a system for monitoring the patient clinical data and allowing to compare the clinical data of the patient with patients showing similar pattern.

### INTRODUCTION

The advent of innovative technologies together with increasing volumes of data being generated for each patient has changed clinical practice. Clinical decisions are now based on large amounts of data from diverse sources and modalities. The medical personnel (MP) or user is now facing the challenge of leveraging available data to ensure clinical and patient management decisions are based on the most relevant and up-to-date data for the condition and are adapted to the patient profile.

### BACKGROUND

Today, medical personnel (MP), including clinicians and staff, can access a variety of clinical, biological and imaging data for their patients. The amount and diversity of information that needs to be considered when making clinical decisions can result in the clinicians' overload. Typically, patients can be seen by multiple clinicians and have multiple medical tests performed through the course of disease and treatment. Oftentimes this medical information is stored across multiple locations and is accessed through multiple interfaces. In addition, for certain conditions, it is also important to consider the evolution of medical test results and patient profile for months and, in some cases, for years. Patient monitoring and clinical decisions thus require accessing and integrating unfiltered and diverse data stored across multiple locations and formats which can be overwhelming and time consuming.

For many diseases there is more than one possible patient management decision and the choice for a specific patient is usually based on their clinical situation, including patient profile and its disease evolution based on the results of multiple different medical tests. Therefore, systems that allow clinical monitoring for patients, providing knowledge and diagnostic specific information that is filtered according to the most relevant guidelines and presented at appropriate times enhance the clinical decision-making process. In addition, such systems should also include functionalities to help the clinicians infer the best follow-up strategy for the patient based of the outcome of patients with similar medical profiles and disease evolution. Display of patient medical history that are static and lack the ability to assess dynamically changes in patient disease evolution alone or in comparison to information from other related patients, render patient management decisions difficult. The ability to dynamically report clinical patient data and ability to define groups of patients with similar characteristics (cohorts) can support patient management decisions.

First-line treatment of stage IV NSCLC requires systemic therapy. Historically, patients were treated with a doublet chemotherapy regimen, such as the combination of a platinum chemotherapy with gemcitabine, vinorelbine or a taxane. In this context, systemic therapy for NSCLC today is selected according to the presence of specific biomarkers. Strong oncogenic driver mutations have been identified in subsets of NSCLC patients in genes such as EGFR, ALK, ROS1, BRAF and NTRK1/2/3. Collectively, these mutations account for approximately 30% of NSCLC cases, and render patients eligible for corresponding specific biomarker-driven targeted therapies. Given that patients eligible for such first-line targeted therapies currently only represent around 30% of all patients with stage IV NSCLC, the vast majority of NSCLC patients cannot benefit from this treatment approach (Arbour and Riely, 2019, JAMA. 322(8): 764-774). For those patients, immunotherapy has dramatically altered the treatment landscape of stage IV NSCLC over the past few years.

Malignancies can overexpress PD-L1 as a mechanism of immune evasion, thereby downregulating the immune response towards the tumor through the inhibitory effects of the PD-L1/PD-1 interaction (programmed cell death (PD)-1 and anti-programmed cell death-ligand 1 (PD-L1)) (Pardoll et al., 2012, Nat Rev Cancer. 12(4): 252-264; Dong et al., 2002, Nat Med. 8(8)793-800). Antibodies directed against PD-1 or PD-L1 can block this interaction, resulting in “releasing the brakes” on the anti-tumor immune reaction. This therapeutic strategy has been successful across many tumor types, including NSCLC (Pardoll et al, 2012, supra).

In this context the immune checkpoint inhibitors anti-PD-(L)1 antibodies pembrolizumab, nivolumab and atezolimumab have been approved by the FDA and EMA as monotherapy regimens in the second-line NSCLC setting after progression on platinum-based chemotherapy (Herbst et al. 2016, Lancet. 387:1540-1550; Borghaei et al., 2015, NEJM, 373:1627-1639; Rittmeyer et al. 2017, Lancet 389:255-265). These second-line studies highlighted two key observations. First, while PD-L1 expression was shown to have some predictive power for response to immunotherapy using immune checkpoint inhibitors in some studies, the predictive value was not comparable to the targeted therapies in patients with specific genomic driver mutations. Second, lung adenocarcinoma patients harboring mutations in EGFR or ALK featured poor responses to immune checkpoint inhibitors compared to wild type tumors (Yang et al., 2020, Annu Rev Med, 71:117-136). In practice, metastatic NSCLC patients that do not have driver mutations amenable to targeted therapies nor contraindications to immunotherapy currently predominantly receive either pembrolizumab monotherapy when PD-L1>50% or pembrolizumab plus chemotherapy combination therapy as first-line treatment. Increasingly, patients with PD-L1>50% may also receive pembrolizumab plus chemotherapy combination therapy, although that practice is still evolving.

Despite the clinical promise of immunotherapy, significant challenges remain as the majority of NSCLC patients seem to be intrinsically resistant to immunotherapy and fail to respond. Overall, only approximately 20-30% of patients treated with immunotherapy show an objective response, although this varies by immune checkpoint inhibitor and clinical setting. At the same time patients are exposed to potentially severe side effects, especially immune-mediated reactions against healthy organs. Additionally, immune checkpoint inhibitors are particularly expensive, with most therapies costing in excess of USD 100,000 annually per patient, constituting a significant financial burden for healthcare systems.

Today, a high level of PD-L1 expression of 50% or above is the only standard predictive biomarker for immune checkpoint inhibitor efficacy as monotherapy in the first-line NSCLC setting (Remon et al., 2020, J Thorac Oncol 15(6):914-947). PD-L1 however remains a suboptimal biomarker of immunotherapy response with several issues limiting its clinical utility. Differences in testing platforms, the use of various cut-off points for expression between different immunotherapy agents and the heterogenous nature PD-L1 expression within tumors have all been points of criticism (Bodor et al., 2020, Cancer 126:260-270). In this context, the predictive power of PD-L1 for immunotherapy response remains limited. Indeed, even NSCLC patients with tumor PD-L1>50% typically only display approximately 45% ORR (overall response rate), and patients with tumor PD-L1>90% still only reach approximately 60% ORR (Aguilar et al., 2019, Ann Oncol. 30:1653-1659).

In that context, it is of critical importance to provide clinicians with tools to support clinical decision making. This could increase the quality of care and enhance patient outcome, help avoid adverse events, while optimizing the resource allocation spend of healthcare systems and both provider and patient satisfaction.

### SUMMARY

This Summary is provided to introduce a selection of concepts in a simplified form that are further described below in the Detailed Description. This Summary is not intended to identify key features or essential features, nor is it intended to limit the scope of the claims included herewith.

One aim of the present invention is to provide the medical personnel or a user with a system for monitoring patient clinical outcome and for supporting clinical decision. The present invention is described by the claim 1.

The present invention proposes monitoring features including a number of advanced features that address unmet needs in the clinical decision-making workflow. The system offers the user the ability to use of cohorts and patient groups built based on the patient's complete clinical profile, medical test results to infer the most appropriate follow-up strategy for the patient. These cohorts can be built by the system using predefined parameters specific to the patient's diagnostic status or condition; or these cohorts can be assembled based on user selected parameters; or these cohorts can be dynamically assembled based on the cohorting parameters defined and used by other users of the application for patients with the same diagnostic status as the patient for which the clinical decision needs to be made. The system further informs the user, via automatic alerts, regarding changes in the patient's cohort (for example new patient added or patient removed) or changes in patient characteristic that occurred during disease evolution, medical test results or changes in the cohort that may impact the cohorting participation of the patient. Alerts can further trigger changes in the information displayed for the patient including for example characteristics of the new cohort that are relevant given the current patient clinical data pertaining to comparison of the characteristics (or inclusion criteria) of the new cohort where the patient could be included relative to the previous patient's cohort.

In addition, the present application, with the claimed system, provides the user with means to visualize all relevant patient information through a unique interface that integrates data from multiple sources and combines knowledge and data to generate and present to the user information that is filtered, organized, and presented according to the diagnostic status of the patient. The system allows access and use of longitudinal information across medical specialties, a feature that is particularly relevant for patient cancer. The system provides an engaging and user-friendly designed interface allowing clinicians to collect and display the results of medical tests and clinical data longitudinally and for different medical specialties. The system further allows the user to access quickly relevant clinical data and gain an overview of the patient medical case, including through integration of multimodal clinical and omics data, a feature that can be used for example during tumor board meetings . The system also contains an interface to predict and report the results of the patient's clinical outcome using available and diagnostic status-specific prediction models that integrate clinical data.

The system also contains a clinical trial matching feature that leverages on the quantitative and qualitative clinical data for the patient.

The system further enables the effective integration of large volumes of clinical data for multiple patients thus providing a complete framework to support clinical research programs.

The system also allows the user to be notified when new events for a patient are added through means of notifications, alerts and communications.

The system supports the following activities for patient monitoring and management by the medical personnel or user, including:
quickly access important clinical data to help manage cancer conditions
provides an engaging interface for clinicians or users to collect and display data longitudinally and across medical specialties
offers a solution for Tumor Board meetings allowing to overview efficiently a patient medical case
display multimodal clinical data and omics data in a combined way for a better understanding of the medical case
the ability to build patient cohorts based on their full clinical profile and then infer the best follow-up strategy for a given patient (in terms of prognostic, diagnosis, and/or treatments)
improve the clinical trial enrolment process with more clinical and omics data, qualitatively and quantitatively
offer a complete platform and framework to Clinical Research Programs that enable such programs to process efficiently a large amount of patient data (clinical and omics data).

A further advantage of the present system is to provide a dynamic visualization of relevant patient information that is updated automatically upon changes in the patient diagnostic status.

Other features and advantages of the present application will be apparent from the following more detailed description of the identified embodiments, taken in conjunction with the accompanying drawings which show, by way of example, the principles of the application.

For that purpose, the present disclosure may provide a computer system for dynamically monitoring clinical outcome, comprising a display screen and connecting at least two databases, at least one database, so called patient database, comprising clinical data of patients including diagnostic status, and a second database, so called display database, comprising a set of display parameters for each diagnostic status, the computer system executing the steps of:
a. Obtaining the clinical data for a patient;
b. Obtaining a set of display parameters based on diagnostic status of the patient;
c. Displaying on a first window the data of the patient according to the set of display parameters, comprising at least:
   i. Clinical patient identifiers of the selected patient;
   ii. Diagnostic status of the selected patient;
   iii. Timeline of selectable events associated with the selected patient;
wherein each selectable event is further displayed in a second window with the detailed metrics of the patient for the selected event.
   The term “database” defines the storage of data in an organized form and can be implemented in various ways. The patient database can be located into a single storage unit or can be spread to numerous storage units. The same physical storage unit can contain the patient's database and the display database. This is also valid for all databases mentioned in the present description (cohort database, reference database).

### BRIEF DESCRIPTION OF THE FIGURES

Objects, aspects, features, and advantages of embodiments disclosed herein will become more fully apparent from the following detailed description, the appended claims, and the accompanying drawing figures in which like reference numerals identify similar or identical elements. Reference numerals that are introduced in the specification in association with a drawing figure may be repeated in one or more subsequent figures without additional description in the specification in order to provide context for other features, and not every element may be labeled in every figure. The drawing figures are not necessarily to scale, emphasis instead being placed upon illustrating embodiments, principles and concepts. The drawings are not intended to limit the scope of the claims included herewith.

The present invention will be better understood with the help of the attached figures.

FIG. 1 illustrates an embodiment of the selection of a patient.

FIG. 2 illustrates a snapshot of the information provided to the user,

FIG. 3 illustrates the distribution of clinical data of a cohort with the highlight of the clinical data of the patient,

FIG. 4 illustrates another example of the distribution of clinical data of a cohort with the highlight of the clinical data of the patient

FIGS. 5 and 6 illustrate an embodiment of a prediction window showing the probability of the evolution of one parameter in view of the evolution of the cohort parameters

FIG. 7 illustrates the process of the selection of the criteria for the creation of a cohort

FIG. 8 to 11 illustrate the process of the selection of the inclusion criteria for the creation of a cohort

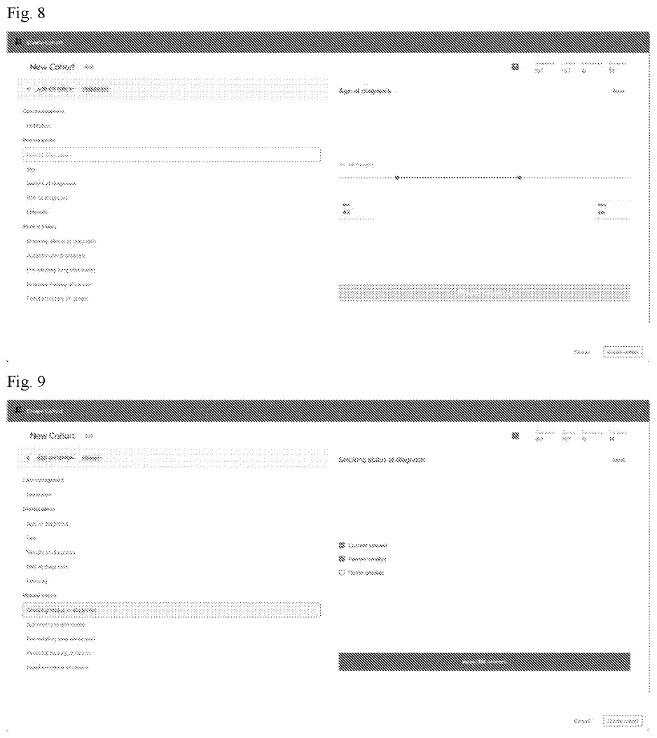

FIG. 12 illustrates the visualization of the cohort by selecting different criteria,

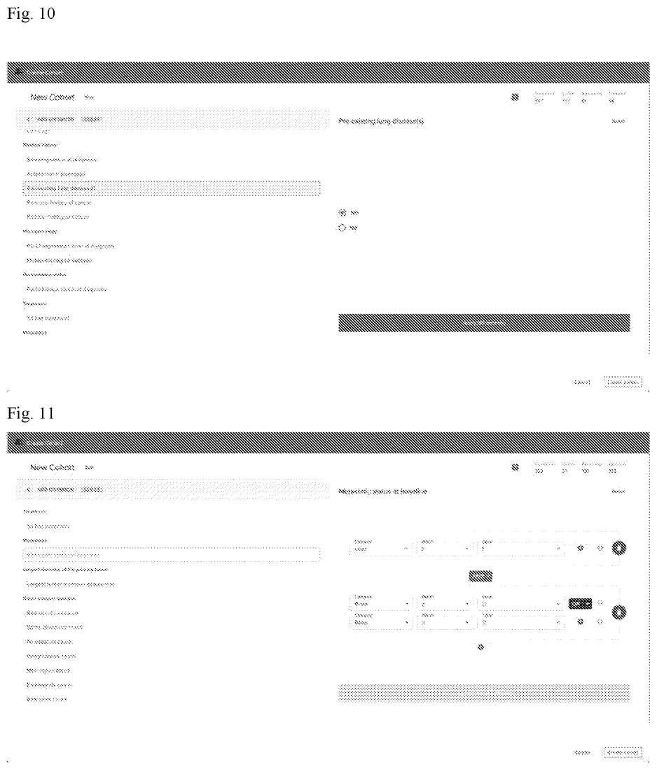

FIG. 13 illustrates a block diagram of a distributed computer system that can implement one or more aspects of an embodiment of the present invention.

FIG. 14 illustrates a block diagram of an electronic device that can implement one or more aspects of an embodiment of the invention.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

It is understood that a “diagnostic status” of a patient refer to the clinical identification of a disease, condition, or injury based on the signs and symptoms a patient is having and the patient's health history and physical exam. In one embodiment the diagnostic status may be a cancer, including but not limited to a brain cancer, a lung cancer, a kidney cancer, and a glioblastoma. In a preferred embodiment, the diagnostic status is a lung cancer, preferably the lung cancer is non-small cell lung cancer (NSCLC), more preferably the lung cancer is a stage IV NSCLC.

It is understood that a “treatment” or a “therapy” refers to any treatment, such as cancer treatment intended to heal, delay or relive the symptoms of a disease. The “first-line treatment” or “primary/initial treatment” or “induction therapy” refers to the initial, or first treatment recommended for a given disease, such as cancer. For example, first-line treatment of stage IV NSCLC may be a pembrolizumab monotherapy, a chemotherapy and pembrolizumab combination therapy, a chemotherapy doublet, such as the combination of a platinum chemotherapy with gemcitabine, vinorelbine or a taxane, and any other suitable treatment. “Second-line treatment” is treatment for a given disease, such as cancer after the first-line treatment has failed, stopped working, or has side effects that aren't tolerated.

It is understood that the patient's response to a treatment may be binary classified as (1) progression (a complete or partial progression) or (2) no progression (a stable disease or absence of progression). Alternatively, the patient's response to a treatment may be classified as (1) a complete response, wherein all the symptoms disappear and there is no evidence of disease; (2) a partial response, wherein the symptoms declined by a percentage, but disease remains; (3) a stable disease wherein the symptoms and the disease don't progress but are not decreasing or (4) progression, wherein the disease has further developed. It is understood that the patient's response to a cancer treatment may be classified as (1) a complete response, wherein all of the cancer or tumor disappears and there is no evidence of disease; (2) a partial response, wherein the cancer has shrunk by a percentage but disease remains; (3) a stable disease, wherein the cancer is neither shrinking nor growing (no change in cancer progression) or (4) progression, wherein there is a progression so that a cancer has further developed. Alternatively, the patient's response to a treatment may be provided as a metric that reflects the probability of the patient's response to the treatment. In addition, confidence ranges may be provided.

The term “Progression-Free Survival (PFS)” refers to the length of time during and after the treatment of cancer, that a patient lives with the disease but it does not progress (assessed as tumour progression, the appearance of new lesions, and/or death).

The term “Overall Survival (OS)” refers to the length of time which begins at diagnosis (or at the start of treatment) and up to the time of death. The PFS and OS are commonly referred to as survival endpoints and measure the efficacy of cancer treatments.

The term “Duration of Response (DoR)” refers to the length of time from response (R) of cancer to a treatment (improvement) to the disease worsening again (progression/death). The DoR is commonly referred to as early efficacy endpoint.

The term “Time-To-Progression (TTP)” refers to the length of time from the date of diagnosis or the start of treatment for a cancer until the cancer starts to get worse or spread to other parts of the body.

It is understood that PFS, OS, DoR and TTP are known endpoints for cancer clinical trials that are time-to-event data and measure the efficacy of cancer treatments.

The term “Response Evaluation Criteria in Solid Tumours (RECIST monitoring)” refers to a set of published rules established by the European Organization for Research and Treatment of Cancer (EORTC), National Cancer Institute (NCI) of the United States and Canadian Cancer Trials Group (CCTG) and pharmaceutical companies. It defines when tumors in cancer patients improve (“respond”), stay the same (“stabilize”), or worsen (“progress”) during treatment. It provides a simple and pragmatic methodology to evaluate the activity and efficacy of new cancer therapeutics in solid tumors, using validated and consistent criteria to assess changes in tumor burden. The RECIST specification establishes a minimum size for measurable lesions, limits the number of lesions to follow and standardizes unidimensional measures. Monitoring parameter, is a clinical parameter used to assess the evolution of the patient disease.

The term “extraction” refers to the signal processing analysis and/or calculation of a quantifiable value, such as a feature, from digital data, such as digital data file record, a digital health database, or a digital signal data input, such as for instance, but not limited to: an image or one or more image segments or elements, or a genomic sequence file or a genomic variant file, or a clinical annotation file, or a biological laboratory report file. Extraction may comprise various processing steps, such as for instance but not limited to: conversion, for instance to a predetermined unit; normalization, for instance to a scalar value in a range between 0 and 1; mathematical operations, for instance adding or multiplying or subtracting or dividing or deriving a value from one or more elements in the digital data to measure a property of the data, for instance the growth of an area or a volume; filtering part or all of the digital data signal, such as cropping, segmenting, extracting subsets such as patterns, regions or volumes of interest, removing elements, such as redundant information; transforming the digital data signal into a different representation space (e.g. from spatial domain to transform domain); digital signal processing analysis methods, or a combination thereof.

The term “aggregation” refers to combining, for instance by concatenation, multiple data inputs into a single data representation.

The term “selection” refers to identifying a subset of data elements into a data set.

The term “prediction” refers to inferring, with a statistical analysis model, the value of an outcome at a future time from the values of a data set at a current time.

### Clinical, Biological, Genomic and Radiological Data

In a preferred embodiment, all the applications of the invention use at least clinical data which could comprise at least one data selected from biological, genomic, and radiological data.

In one embodiment, at least clinical data are used in the applications as described herein, wherein a computer-implemented system to monitor disease is used for a patient with kidney cancer.

In one embodiment, at least clinical data are used in the applications as described herein, wherein a computer-implemented system is used to monitor disease for a patient with brain cancer.

In one embodiment, clinical data are used in methods as described herein, wherein a computer-implemented system is configured to monitor disease, such as a patient with non-small cell lung cancer (NSCLC), particularly a patient with stage IV non-small cell lung cancer.

In one embodiment, the multimodal data may be obtained or generated from one or more sources. This may include health care providers, e.g., hospitals, primary care units, third parties providing services for medical data analysis and storage. For example, clinical data may be obtained from databases that may be retrieved from electronic medical records (EMRs), electronic health records (EHRs), personal health records (PHRs) or an electronic case report form (eCRF). Clinical data may be obtained from Laboratory Information Management System (LIMS). Radiological data may be obtained from Picture Archiving and Communication System (PACS). Genomic data may be obtained from any system that stores genetic sequences, e.g. Next Generation sequencing (NGS) FastQ file or a genomic variant list in an NGS Variant Call Format.

The category definition and exemplary content of clinical, biological, genomic, and radiological data are described herein. Further, data may be defined based on the example of a lung cancer.

### Clinical Data

Clinical data for any patient may include, but is not limited to the patient's:
demographics such as gender, age (month and year of birth), ethnicity, height and weight;
medical history such as smoking status, personal history of diseases, previous familial history of diseases;
disease history such as date of start of disease, disease stage, performance status at diagnosis, treatment history, hospitalization and/or death and organs affected, performance status and clinical response at first/further evaluation, progression status, including date and site of progression, treatment status after progression and/or vital status at the last available instance.

Clinical data for the cancer patient may include, but is not limited to the patient's:
demographics such as gender, age (month and year of birth), ethnicity, height and weight;
medical history such as smoking status, personal history of autoimmune diseases, pre-existing disease, previous familial history of cancer and previous personal history of cancer;
disease history such as date of cancer, cancer stage, cancer subtype (IVA, IVB), performance status at diagnosis, corticosteroids and antibiotics treatment history, therapy received and number of cycles by progression and/or in total, presence of treatment toxicity leading to treatment discontinuation, hospitalization and/or death and organs affected, performance status and clinical response at first/further evaluation, progression status, including date and site of progression, treatment status after progression and vital status at last available instance.

Clinical data for the patient with stage IV NSCLC diagnosis may include, but is not limited to the patient's:
demographics such as gender, age (month and year of birth), ethnicity, height and weight;
medical history such as smoking status, personal history of autoimmune diseases, pre-existing lung disease, previous familial history of cancer and previous personal history of cancer;
disease history such as date of stage IV NSCLC diagnosis and subtype (IVA, IVB), performance status at stage IV NSCLC diagnosis, corticosteroids and antibiotics treatment history, therapy received and number of cycles by progression and/or in total, presence of treatment toxicity leading to treatment discontinuation, hospitalization and/or death and organs affected, performance status and clinical response at first/further evaluation, progression status, including date and site of progression, treatment status after progression and vital status at last available instance.

In one embodiment, the clinical data may further comprise a treatment plan comprising date of treatment (if available), name of medication, number of completed treatment cycles, toxicity observed (if any), date of start and end of treatment (depending on existence of past treatment and if available). Any additional medication intake (e.g. antibiotics and corticosteroids) may be also displayed.

In one embodiment, the clinical data may further comprise risk factors including but not limited to smoking status, alcohol consumption, obesity, other lifestyle factors. The exact nature of risk factors may vary by disease type.

In one embodiment, the clinical data for a patient with a lung cancer, in particular with stage IV NSCLC, may comprise at least one data aspect of the patient's age, Eastern Cooperative Oncology Group (ECOG) performance status (PS), autoimmune diseases history, steroid treatment history, gut microbiome status, antibiotics treatment history, disease history (such as liver metastasis, brain metastasis and bone metastasis) and immune-related adverse events.

In one embodiment, the clinical data for a patient with a lung cancer, in particular with stage IV NSCLC, may comprise at least one of the following: gender, age (month and year of birth), ethnicity, height, weight, smoking status, personal history of autoimmune diseases, pre-existing lung disease, previous familial history of cancer, previous personal history of cancer, date of first NSCLC diagnosis, stage of first NSCLC diagnosis, date of stage IV NSCLC diagnosis, stage IV subtype at diagnosis (IVA or IVB), performance status at diagnosis, corticosteroids treatment received less than 12 months before stage IV NSCLC diagnosis, antibiotics treatment received less than 1 month before stage IV NSCLC diagnosis, start date of treatment with pembrolizumab, pembrolizumab and chemotherapy combination, or chemotherapy doublet, pembrolizumab dosing scheme, chemotherapy doublet regimen, number of cycles of therapy received by first evaluation, number of cycles of therapy received by progression, presence of treatment toxicity leading to treatment discontinuation, hospitalization, or death and organs affected, performance status at first/further evaluation, clinical response at first/further evaluation, progression status at last available update, date and site of progression, treatment status after progression, second-line therapy received, date of and viral status at last update, cause of death.

Clinical data refers to information that may also comprise descriptive data such as patients' response to the treatment status, patient's disease progression. The descriptive data may be further categorized such as, for example, the patient's response to the treatment may be classified as a complete response, a partial response, a stable disease or a progression, wherein the patient's disease progression may be classified as an increased growth speed and invasiveness of the tumor cells. This classification may be assigned numerical variables at the pre-processing step. The term “multimodal data” or “multiomics data” refers to a set of at least two types of data selected from clinical, biological, genomic and radiological data. Any combination of at least two types of data selected from clinical, biological, genomic and radiological data can be referred to as multimodal or multiomics data. The possible sets may thus comprise or consist of: clinical and biological data; clinical and genomic data; clinical and radiological data; biological and genomic data; biological and radiological data; genomic and radiological data; clinical, biological and genomic data; clinical, biological and radiological data; biological, genomic and radiological data; clinical, genomic and radiological data; and clinical, biological, genomic and radiological data.

### Biological Data

The clinical data can further comprise “biological data” for the patient and may include but is not limited to the patient's: disease type and stage, expression level of relevant disease genes, such as receptors, blood analysis at baseline and first/further evaluation (hematology and biochemistry). Blood analysis results may comprise red-blood cell count, white blood count and/or biochemistry. In a preferred embodiment, the blood analysis results comprise white blood cells count, neutrophils count, lymphocytes count, monocytes count, eosinophils count, basophils count, platelets count, red blood cells count, hemoglobin levels, LDH levels, albumin, CRP levels.

Biological data for the cancer patient may include but is not limited to the patient's: cancer stage and histopathology type at diagnosis, expression level of relevant receptors, blood analysis at baseline and first/further evaluation (hematology and biochemistry).

Biological data for the patient with stage IV NSCLC diagnosis may include but is not limited to the patient's stage IV NSCLC histopathology type at diagnosis, PD-L1 expression level, immunohistochemistry antibody used for PD-L1 measurement, blood analysis at baseline and first/further evaluation (hematology and biochemistry).

In one embodiment, the biological data for a patient with a lung cancer, in particular with stage IV NSCLC, may comprise data on PD-L1 expression on tumor cells.

In one embodiment, the biological data for a patient with a lung cancer, in particular with stage IV NSCLC, may comprise at least one data on neutrophil-to-lymphocyte ratio, enzyme lactate dehydrogenase (LDH) levels and/or blood TMB (bTMB).

In one embodiment, the biological data for a patient with a lung cancer, in particular with stage IV NSCLC, may comprise at least one or consist of the following: stage IV NSCLC histopathology at diagnosis, PD-L1 expression level, immunohistochemistry antibody used for PD-L1 measurement, date of blood analysis at baseline, white blood cells count at baseline, neutrophils count at baseline, lymphocytes count at baseline, monocytes count at baseline, eosinophils count at baseline, basophils count at baseline, platelets count at baseline, red blood cells count at baseline, hemoglobin levels at baseline, LDH levels at baseline, albumin levels at baseline, CRP levels at baseline, date of blood analysis at first/further evaluation, white blood cells count at first/further evaluation, neutrophils count at first/further evaluation, lymphocytes count at first/further evaluation, monocytes count at first/further evaluation, eosinophils count at first/further evaluation, basophils count at first/further evaluation, platelets count at first/further evaluation, red blood cells count at first/further evaluation, hemoglobin levels at first/further evaluation, LDH levels at first/further evaluation, albumin levels at first/further evaluation, CRP levels at first/further evaluation.

Biological data for the patient may include digital pathology data and proteomic data.

### Genomics Data

The clinical data of a patient can further comprise “genomic data” which refers to a digital representation of genomic information, such as a DNA sequence. The term “genomic data” may be viewed as including “molecular data”. In a next generation sequencing (NGS) bioinformatics workflow, genomic data may refer either to a raw nucleotide DNA sequence out from a sequencer (FASTQ file format), and/or to an aligned nucleotide sequence relative to a reference genome (BAM or SAM file format), and/or to a list of variants out from a variant calling step (VCF file format), and/or a list of annotated variants out of a variant annotation step (VCF file format).

A “variant” or a “genomic variant” refers to genomic sequence differences relative to a reference sequence. In bioinformatics data processing, a variant is uniquely identified by its position along a chromosome (chr, pos) and its difference relative to a reference genome at this position (ref, alt). Variants may include single nucleotide permutations (SNPs) or other single nucleotide variants (SNVs), insertions or deletions (INDELs), copy number variants (CNVs), as well as large rearrangements, substitutions, duplications, translocations, and others. In a bioinformatics secondary analysis workflow, a variant caller may apply variant calling to produce one or more variant calls listed in a Variant Calling File (VCF format). A germline variant is a variant inherited from at least one individual parent that differs from the wildtype genomic value as registered in a reference database, and that is present in all normal cells of the individual. A somatic variant or a somatic mutation or a somatic alteration is a variant caused by a genomic alteration, that is present in one or more somatic cells of the individual, for example in tumor cells.

A “mutation” or a “mutated gene” refers to a gene for each at least one variant has been identified. A “mutated gene status” may be classified as mutated in the latter case or normal otherwise. This status is routinely used as a biomarker in cancer diagnosis and prognosis. For instance, the ALK gene mutation or the EGFR gene mutation have been shown of particular relevance in relation with lung cancer.

A “mutational load” or “mutation load” or “mutation burden” or “mutational burden”, or for a tumor a “tumor mutational burden” or “tumor mutational load” or “TMB” refers to a biomarker measured as the number of somatic mutations per megabase of an interrogated genomic sequence.

A “MSI status” or “Microsatellite Instability status” or “Micro satellite instability status” refers to the status of a genomic alteration due to insertions or deletions of a few nucleotides in the microsatellite repeat regions based upon one nucleotide repeat (homopolymers) or a few nucleotides (heteropolymers), due a DNA mismatch repair system deficiency. This status is routinely used as a biomarker in cancer diagnosis and prognosis, and in particular in uterine, colon and stomach cancers such as UCES (Uterine Corpus Endometrial Carcinoma), COAD (Colon Adenocarcinoma) and STAD (Stomach adenocarcinoma). The MSI status of genomic alterations for a patient is usually categorized as:
Micro-satellite stable, MSS: no evidence of any of the biomarker genomic loci exhibiting instability.
Micro-satellite instable-low, MSI-L: evidence of instability in only one marker locus.
Microsatellite instable-high, MSI-H: evidence of instability in at least two marker loci.

A “homologous recombination deficiency status” or “HRD status” refers to a classification of homologous recombination pathway and relates to any cellular state/event that results in homologous recombination pathway deficiency. HRD status may be classified as positive (HRD+) wherein a homologous recombination pathway is deficient or may be classified as negative (HRD−) wherein a homologous recombination pathway is not deficient or may be classified as undetermined otherwise (HRD uncertain, HRD unknown).

A “genomic pathway” or a “genetic pathway” refers a set of genomic loci or gene expression data which are significantly impacted in a given condition. In a bioinformatics secondary analysis workflow pathway analysis approaches use available pathway databases and the given genomic data or gene expression data from a patient to identify the presence or an absence of a genomic pathway for this patient.

Genomics data for the patient may include, but is not limited to the patient's disease site mutational status such as obtained through NGS VCF file. The patient's disease site may be a tumor or genetic material released by tumor and found in the blood.

Genomics data for the cancer patient may include but are not limited to the patient's cancer mutational status such as obtained through NGS VCF file.

In one embodiment, the genomics data for a patient with a lung cancer, in particular with stage IV NSCLC, may comprise at least one or consist of the following: EGFR and ALK mutational status. Tumor mutational status may be obtained through any known methods such as through NGS VCF file (based on locally available NGS panels), sanger sequencing, immunochemistry and the like.

In one embodiment, the genomics data for a patient with a lung cancer, in particular with stage IV NSCLC, may comprise data on tumor cell mutations such as at least one mutational status of EGFR, ALK, KRAS, STK11/LKB1, KEAP1, PTEN, PIK3CA, TP53, ROS1, BRAF, NTRK1/2/3, components of the DNA repair pathways such as including mismatch repair genes, POLE and BRCA2, components of the interferon-gamma (IFN-gamma) signaling such as including loss of function mutations in JAK1, JAK2 and beta-2-microglubulin (B2M).

In one embodiment, the genomics data for a patient with a lung cancer, in particular with stage IV NSCLC, may comprise data on tumor immunogenicity, such as tumor mutational burden (TMB), microsatellite instability (MSI) and defective mismatch repair (dMMR).

It is understood that genomics data may be collected only once at baseline or first or further evaluation or collected at multiple instances.

### Radiological/Radiomics Data

The clinical data of a patient can further comprise “radiological data”, also referred to as radiomics data, and may be collected images and may include, but are not limited to computerized tomography (CT), positron emission tomography (PET), PET/CT, magnetic resonance imaging (MRI), single-photon emission computerized tomography (SPECT), and the like.

Radiological data for the patient may include, but is not limited to the patient's imaging at pre-baseline, at baseline, at first/further evaluation.

The terms “medical image data” or “radiological data” or “imaging data” refer to the digital images data and, in particular, one or more images collected for a patient at any time during diagnosis and/or treatment. These images may be acquired from the patient examination in one or more medical centers in charge with one or more imaging modality such as CT, PET, MRI, SPECT, and others. These images may be in 2D or 3D format. These images may be securely collected, stored, archived, and transmitted to the radiomics processing system of the invention in accordance with the PACS (Picture Archiving and Communication System) and DICOM (Digital Imaging and Communications in Medicine) digital medical imaging technology standards that are widely deployed in healthcare organizations worldwide.

Radiological data for the cancer patient may include, but is not limited to the patient's: imaging at pre-baseline if available (millimetric injected CT cancer site scan at portal time, slices <3 mm), imaging at baseline (millimetric injected CT cancer site scan at portal time, slices <3 mm; PET/CT, CT, MRI if available), imaging at first/further evaluation (millimetric injected CT cancer site scan at portal time, slices <3 mm; CT, MRI if available, imaging during follow-up visits after first/further evaluation if available, imaging at progression (millimetric injected CT cancer site scans at portal time, slices <3 mm; CT, MRI if available), number of metastasis for each metastatic site at baseline and at first/further evaluation, RECIST (Response Evaluation Criteria in Solid Tumors) criteria if available.

In one embodiment, the radiological data for a patient with a lung cancer, in particular with stage IV NSCLC, may comprise data on imaging-based assessment of clinical tumor burden.

Radiological data for the patient with stage IV NSCLC diagnosis may include, but are not limited to the patient's: imaging at pre-baseline if available (millimetric injected CT thoracic, abdomen and pelvis scans at portal time, slices <3 mm), imaging at baseline (millimetric injected CT thoracic, abdomen and pelvis scans at portal time, slices <3 mm; PET/CT, brain CT, brain MRI if available), imaging at first/further evaluation (millimetric injected CT thoracic, abdomen and pelvis scans at portal time, slices <3 mm; brain CT, brain MRI if available), imaging during follow-up visits after first/further evaluation if available, imaging at progression (millimetric injected CT thoracic, abdomen and pelvis scans at portal time, slices <3 mm; brain CT, brain MRI if available), number of metastasis for each metastatic site at baseline and at first/further evaluation, RECIST criteria if available.

In one embodiment, the radiological data for a patient with a lung cancer, in particular with stage IV NSCLC, may comprise at least one of the following: pre-baseline chest CT scan availability and date, baseline computed tomography of thorax, abdomen and pelvis (CT-TAP) scan date, baseline CT-TAP RECIST (if available), baseline brain CT scan availability and date (if available), baseline PET/CT scan availability and date (if available), baseline brain MRI availability and date (if available), extent of metastatic load assessment through imaging at baseline, extent of metastatic disease at baseline-status of affected organs, first/further evaluation chest CT scan date, first/further evaluation chest CT scan RECIST (if available), first/further evaluation CT-TAP scan availability and date (if available), first/further evaluation brain CT scan availability and date (if available), first/further evaluation brain MRI availability and date (if available), follow-up post-first/further evaluation chest CT scan date (if available), follow-up post-first/further evaluation chest CT scan RECIST (if available), follow-up post-first/further evaluation CT-TAP scan availability and date (if available), follow-up post-first/further evaluation brain CT scan availability and date (if available), progression evaluation chest CT scan date, progression evaluation chest CT scan RECIST (if available), progression evaluation CT-TAP scan availability and date (if available), progression evaluation brain CT scan availability and date (if available).

### Other Modalities

Any omics data modality that can provide insights into patient diagnostic status and clinical outcome can also be used in and integrated in the described system. A non-exhaustive list includes transcriptomics, epigenomics, metabolomics, metagenomics, pharmacogenomics, spatial genomics.

The clinical data as described above are labelled as clinical parameters. The term “clinical parameter” means the name or label or description of a parameter for a given patient, the clinical data being the current value for this parameter.

### Prediction and Inputting Missing Data

A “machine learning model” refers to a data model or a data classifier which has been trained using a supervised, semi-supervised or unsupervised learning technique as known in the data science art, as opposed to an explicit statistical model. The data input may be represented as a 1D signal (vector), a 2D signal (matrix), or more generally a multidimensional array signal (for instance a tensor, or a RGB color image represented as 3*2D signals of its Red, Green and Blue color decomposition planes—3 matrices), and/or a combination thereof. A multidimensional array is mathematically defined by a data structure arranged along at least two dimensions, each dimension recording more than 1 value. In the case of a deep learning classifier, the data input is further processed through a series of data processing layers to implicitly capture the hidden data structures, the data signatures and underlying patterns. Thanks to the use of multiple data processing layers, deep learning facilitates the generalization of automated data processing to a diversity of complex pattern detection and data analysis tasks. The machine learning model may be trained within a supervised, semi-supervised or unsupervised learning framework. Within a supervised learning framework, a model learns a function to map an output result from an input data set, based on example pairs of inputs and matching outputs. Examples of machine learning models used for supervised learning include Support Vector Machines (SVM), regression analysis, linear regression, logistic regression, naïve Bayes, linear discriminant analysis, decision trees, k-nearest neighbor algorithms, random forest, artificial neural networks (ANN) such as convolutional neural networks (CNN), recurrent neural networks (RNN), fully-connected neural networks, long short-term memory (LSTM) models, and others; and/or a combination thereof. A model trained within an unsupervised learning framework infers a function that identifies the hidden structure of a data set, without requiring prior knowledge on the data. Examples of unsupervised machine learning models known in the art include clustering such as k-means clustering, mixture model clustering, hierarchical clustering; anomaly detection methods; principal component analysis (PCA), independent component analysis (ICA), T-distributed Stochastic Neighbor Embedding (t-SNE); generative models; and/or unsupervised neural networks; autoencoders; and/or a combination thereof. Semi-supervised learning (SSL) is a machine learning framework within which one can train a model using both labeled and unlabeled data. Data augmentation methods can be optionally used to produce artificial data samples out of a scarce set of real data samples and increase the number and diversity of data used for model training. Unlabeled data, when used in conjunction with a small amount of labeled data, can produce considerable improvement in learning accuracy compared to other frameworks. This approach is particularly interesting when only part of the available data is labeled.

The machine learning model may be also trained to impute patient's missing features and may be referred to as an imputation machine learning model. A machine learning model for imputing a patient's missing features for use in the computer-implemented method of predicting treatment response or treatment efficacy of a patient according to the invention may be trained by comprising inputting to a machine learning supervised training algorithm a set of features comprising at least two types of features selected from clinical, biological, genomic and radiological features of cohort of patients having the same disease and receiving the same treatment as the patient for whom the prediction is performed. The machine learning model for imputing a patient's missing features is trained to produce at its output a complete list of features for a patient from an incomplete list. In one embodiment, the step of imputing missing features is performed when the patient's multimodal features are at least 60% complete, at least 65% complete, at least 70% complete, at least 75% complete, or preferably at least 75% complete. Percentage of completeness of data may be calculated as relative to the complete set of features that can be extracted for the data from the patient.

In one embodiment, the computer system of the invention detect that the patient's multimodal features are below 60% complete for executing a prediction and inform the user that by adding a specified data or requesting specific analysis, the user may have a prediction of the clinical outcome.

In one embodiment, the computer system of the invention displays a prediction of clinical outcome, wherein the prediction is complemented by a report with the list of informative features identifiers used for the prediction machine learning model training, or treatment features' relative contribution or weights used in the method of predicting treatment response or efficacy of a patient. In one embodiment, the prediction is made earliest at first evaluation time and the prediction of treatment response or treatment efficacy of a patient is for a subsequent evaluation time, such that the prediction may be made at second evaluation time for a third evaluation time, and the like combinations.

### Operations

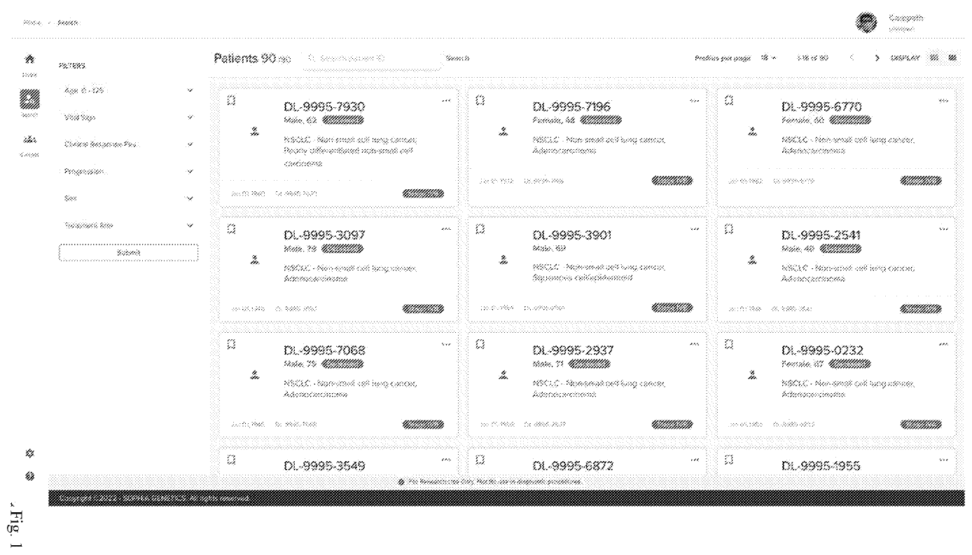

The first action of the user or medical personnel is to select a patient. FIG. 1 displays a first window in which several patients are displayed for selection. In the example of FIG. 1, only the reference of a patient is visible, the name may remain hidden. This may be the preferred presentation in order to avoid leakage of sensitive information.

The presentation is also useful for study purpose for example to select one patient with a specific pattern. In a quick glance, the user can select a case of interest. In one embodiment of the invention, where a change of diagnostic status has been detected, the computer-system highlight the patient for whom change of diagnostic status has occurred.

The selection of a patient may be executed by simply clicking on one of the labels or via a selection menu, by typing the name of the patient or entering other data allowing to identify the patient (such as insurance number).

The patient's data may be stored in a patient database containing the various clinical data of the patient such as diagnostics, treatments, analysis, images, and/or other aspects generated for this patient. Each clinical data is stored with respect of a clinical parameter.

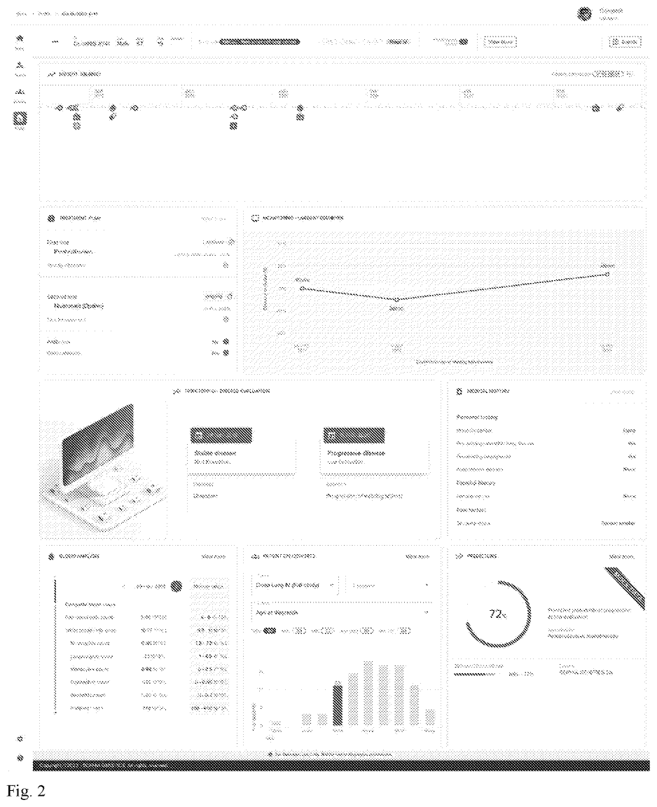

In FIG. 2, the timeline of selectable events is displayed on a first window. As a non-limiting example, the X reference is the time and all events related to a particular patient may be marked at the time when the event occurs. For each event, a symbol may display the nature of the information. While the cursor is hovering above the symbol, a text description may appear in another window. It may then be possible to click on one event and thus open another window for displaying more details about the selected event. In one embodiment, the events displayed in the timeline of event are selected by the user. In a preferred embodiment, the events displayed in the timeline are selected by the computer-system according to the diagnostic status. Selectable event-its a medical/clinical event of the patient that occurred to the patient and is recorded in the clinical database mapped to the timeline of events displayed for the patient. The selectable event can be selected using the GUI to gain more detailed information on the clinical data associate with the event.

In one embodiment, the timeline is not linear and may be interrupted to better grasp the events. As a non-limiting example, a first series of treatments were carried out in 2020 and none in 2021. In such an example, further treatments were applied in 2022. The first left portion of the window may show the events in 2020 and the right portion of the window may show the events in 2022. In an embodiment, the timeline is interrupted in the middle by a sign “//” to inform the reader that the timeline is interrupted.

While the user clicks on one event, the data related to this event may be displayed in a new window with the detailed metric(s) of the patient for the selected event.

The current treatment may be displayed on a second window under the title “Treatment Plan”. In one embodiment, the second window displays details of the Treatment Plan and may comprise therapy type (e.g., surgery, radiotherapy, pharmaceutical therapy, other), medication used in case of pharmaceutical therapy, dosage or dose prescription as suitable, number of cycles/fractions received as suitable, start date, as suitable, end date as suitable, dates of all specific therapy events as suitable. A third window may be dedicated to the result of the imagery, for example, showing the evolution of a tumor.

At the top of this screen, the patient identifier is shown as well as the diagnostic status. In one embodiment of the invention, the patient identifier comprises Patient ID, Age, Gender, current diagnostic status, last weight, last height and/or Body Mass Index (BMI). In a further embodiment of the invention, latest performance status is also shown with the patient identifier at the top of the screen.

The second middle window may display the blood analysis result of the patient at a specific date (as indicated in the upper part of the window). In an embodiment, in addition to the results of the patient, a column shows what should be the normal range. In a preferred embodiment, each time that a value is outside the normal range, the value of the patient is highlighted. In one embodiment of the invention, the blood results comprise white blood cells count, neutrophils count, lymphocytes count, monocytes count, eosinophils count, basophils count, platelets count, red blood cells count, hemoglobin levels, LDH levels, albumin, CRP levels. In another embodiment of the invention, the user may see blood results as measured at another time point by clicking at the clickable arrowhead next to the date. Such clicking may retrieve and display the blood results of the previous or next timepoint.

In addition to the two aforementioned windows, another window (for example, the right window) may be displayed if the patient is part of a cohort. A cohort is a group of individuals who share a common trait, such as birth year. In medicine, a cohort is a group that is part of a clinical trial or study and is observed over a period of time. More generally the cohort may be a set of patients linked with a common denominator related to the demographic data or a health status. In the present example, the selected patient is compared with the cohort defined by the patients having a lung cancer in phase 4 (Deep Lung IV) and for any type of treatments and all ages.

### Cohorting

A ‘cohort’ is defined by a set of criteria applied on the clinical data. The cohort is linked by a predefined criteria, or range of criteria, of at least one clinical parameter of a patient. A patient cohort can be defined by identification of patients with similar characteristics or based on a set of inclusion criteria. A cohort is defined by a set of cohorting clinical parameters for which the cohorting inclusion criteria has a specific value. Those criteria can include a diversity of data including patient demographic data (e.g. birth, death, entry or exit of a clinical study), diagnosis status, patient performance status, genomic, clinical, biological or radiomics features, toxicity, disease progression, risk factors or any monitoring parameter, histopathology type, or depend on a one or more thresholds defined based on patient data.

‘Cohorting’ refers to the process of assembling a cohort based on a number of shared characteristics or a set of inclusion criteria. Cohorting may be performed automatically and for a given diagnostic status based on a set of pre-defined inclusion criteria. These criteria are applied on the clinical data of the patients and are referred as “cohorting inclusion criteria”. Alternatively, the user may define cohorting criteria based on available patient parameters.

According to one embodiment, the computer system of the present disclosure may comprise a cohort database comprising a plurality of cohorts, each cohort being represented by a set of cohorting parameters selected among the clinical parameters, each cohorting parameter having a predefined cohorting inclusion criteria, said system being configured to execute the steps of:
Selecting, from the patient database, a group of patients to study for which the clinical data match (or is included) a set of study inclusion criteria of a set of study clinical parameters,
defining a set of cohorting inclusion criteria of a set of cohorting clinical parameters including the set of study clinical parameters and associated inclusion criteria, said set of cohorting parameters and the inclusion criteria representing a cohort,
parsing the clinical data of the patient's database to retrieve the list of patients part of the cohort,
Storing the cohorting clinical parameters and the inclusion criteria for the cohort in the cohort database,
Storing in the cohort database the list of patients part of the cohort.

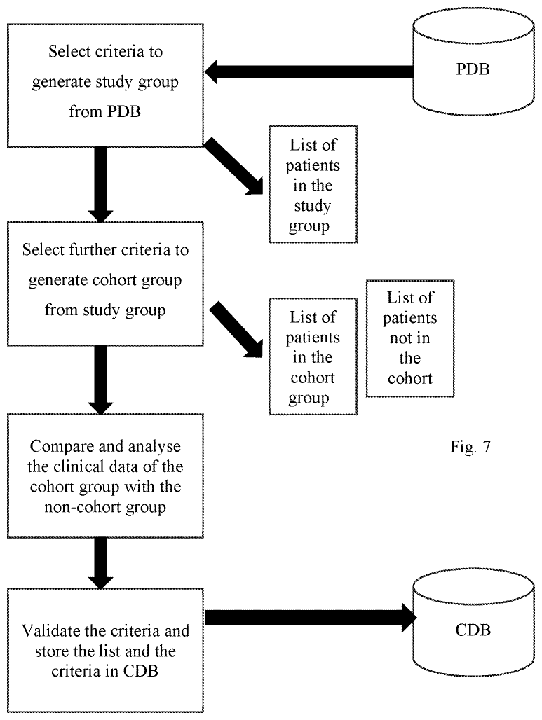

The process of creating a new cohort is a two steps process. The FIG. 7 illustrates this process. The first step is the definition of a study group of patients linked by a common denominator. This denominator is called the “study inclusion criteria”. It could be for example all patients having a specific diagnostic and age range. These criteria form the study parameters (e.g. diagnostic and age) with a specific study inclusion criteria (e.g. diagnostic=lung cancer and age=range between 40 to 50). Once these criteria for the study group of patients are defined, a first parsing process can take place on the patient's database PDB. The patient database PDB contains the clinical data of the patients. As explained above, each patient is identified by a patient identifier. This step creates a list of patients part of the study group. The clinical data of the patients in the patient database PDB are compared with the study inclusion criteria of the study group to extract the list of patients matching these criteria. “Matching” means that the clinical data has the same value as the criteria or within the range defined by the criteria. The second step is to refine the criteria and add more criteria. A set of cohorting parameters are defined which include the first set of study parameters defining the study group. A set of cohorting inclusion criteria are defined as criteria to parse the patient's database and to extract the list of patients part of the new cohort. Alternatively, the system can parse only the list of patients of the study group with the additional criteria defining the cohort. The system generates a list of patients included in the cohort and a list of patients not in the cohort but in the study group (see FIG. 7). The number of patients in these two lists are displayed and the comparison of these numbers indicates to the user the pertinence of the new cohort. The system also can display the clinical data of the patients included in the cohort and of the patients not in the cohort but in the study group. Once validation by the user, the set of cohorting parameters with the cohorting inclusion criteria are stored in the cohort database CDB. The cohort database stores also the current list of patients part of the cohort for future comparison. The list of patients is represented by the list of the patient's identifier.

In view of the aggregation of different databases, it is possible that some patient's parameters are not known for some patients. The clinical data is left blank for some parameters. In this case, according to one embodiment, the study group is filtered to have only the list of patients for which the clinical data of the cohorting clinical parameters is known. This way, the number of the study group is more accurate and the comparison between the number in the study group and the number of the patients in the cohort (called “cohort group”) is more accurate. The number of patients of study group and the number of patients in the cohort group are displayed and can be compared. Further information can be displayed such as the clinical data corresponding to the cohorting clinical parameters for the non-cohort group and the cohort group.

For a better accuracy, a “non-cohort group” is defined by patients part of the study group but not in the cohort group. It is therefore possible to analyse and compare the clinical data of the non-cohort group with the clinical data of the cohort group.

The system comprises processing means to compare and display, for the cohorting parameters, the clinical data of the patients within the new cohort with the clinical data of the study group. This comparison may be completed by the display of a comparison metric representing statistical significance of the difference between the clinical data of the patients within the cohort and the clinical data of the group of patients not part of the cohort. One method is to use the p-value representing the statistical significance of the difference between the clinical data of the patients within the new cohort and the clinical data of the study group (or the non-cohort group).

In this embodiment, a cohort database is connected to the system and comprises a list of patients part of the cohort. Each cohort may be linked by a set of cohort clinical parameters and inclusion criteria and the patients fitting into these criteria may be part of the cohort.

The computer system may then be configured to determine a statistical representation of the cohort representing the proximity of the patient's clinical data with the clinical data of the patients part of the cohort. In such an embodiment, a metric is thus produced for each cohort and the computer system may display on a window the result of the evaluation, showing, for example, through a gaussian representation, each cohort in X and the metric related to this cohort in Y.

The application may support cohorting for a plurality of patients that belong to the user account (i.e., the medical institution or department of the institution of the user). In addition, the application may also support inclusion of a plurality of patients from other accounts (i.e., other medical institution or department of the institution). These patients may be further filtered to restrict inclusion for example to, certain hospitals or medical centers, clinical research consortiums or geographic locations. The list of patients in a cohort may be updated, automatically by the application, when new patient or new patient data is available and matches (or is included in) the set of inclusion criteria of any cohort. The system may support user notification when a patient automatically enters or exits any cohort. Further details will be given below.

In an embodiment, the application also supports cohorting in the context of a clinical study to follow patient inclusion and offers specific statistical analysis tools.

The application may allow comparison of results for the cohort to results of other cohorts, including clinical outcome including comparison of clinical outcome and descriptive statistical analysis. The comparison may also include statistical comparison estimations such as p-value or hazard ratio.

The application may allow monitoring of more than one cohort simultaneously to support patient management decisions for one or several patients. The application may be configured to support clinical decision making for patients with the same characteristics simultaneously. This can be used for managing patient groups within one institution or in the context of a clinical study, for example. In an embodiment, the application further allows the user to follow up with these cohorts using cohort analysis tools specific to the study, for example creation of sub-cohorts based on a subset of cohorting parameters from the clinical study cohort and comparison of these sub-cohorts outcome and descriptive statistical analysis to the other patients of the study. The creation of a sub-cohort from a main cohort is based on using the same cohort parameters and inclusion criteria of the main cohort with at least one additional parameter, thus narrowing the criteria and reducing the number of patients in the sub-cohort.

The system may offer the possibility to link a particular cohort set of parameters to therapeutic guidelines so that the guideline or clinical recommendation is automatically proposed to the clinician when a patient clinical data matches (or is included in) the cohort inclusion criteria. The application also may allow the user to compare the characteristics of a patient or a plurality of patients with therapeutic guidelines, for example, those provided by the National Comprehensive Cancer Network to identify patients that are eligible for a particular treatment. In a further embodiment, the user may compare patient and cohort information for each of the guideline criteria and anticipate potential clinical management follow up decisions.

Cohorts may either be predefined and stored or be assembled de novo based on the stored cohorting parameters. The system supports assembling cohorts using data for a plurality of patients linked to the user account. In addition, the system may also support inclusion of a plurality of patient clinical data available from other accounts. Accordingly, these patients may be further filtered to restrict inclusion, for example, to certain hospital or medical centers, clinical research consortiums, or geographic locations.

Cohorts and cohorting features may be shared via the applications across different users or user groups to support multicentric studies or other analysis. User groups can be part of different medical institutions and cohorts, associated patients clinical and personal information can be shared via the application provided agreement between user groups. The complete data that can be shared for the patients includes but is not limited to:
Patient data, extracted features, computed metrics, cohort information, prediction results;
personal patient information (name, birthdate, hospital identifier);
patient medical data (medical events, test results, imaging data);
The information that this patient is currently handled by a different institution than the institution of the user;
The information that this patient medical case is accessible in read-only mode;
The identity of the user that gave the user group access to this case;
The information that the patient gave his consent for sharing his medical data with the medical institution.

### Smart Cohort Management

The present system may be configured to group patients in cohorts defined by a set of inclusion criteria as explained before. Accordingly, the system may include features to edit, delete, and/or duplicate cohorts. Provided that access is authorized for an existing cohort, the system may be configured to perform the following actions on the cohorts:
Edit: modify cohorts name, description an inclusion criteria,
Duplicate: create a copy of a cohort, and/or
Delete: Remove a cohort from my account for any user of the account.

Such actions may be initiated on a client device but may be executed and processed on a server. For example, in an embodiment where the application is hosted on an application hosting server, the application hosting server may be configured to edit, duplicate, and/or delete the cohort as a function of instructions received from the client device.

### Additional Information

The Cohort May Be Defined by at Least:
an identifier
a name
scope (for example, account versus network)
a set of cohorting clinical parameter having a specified cohorting inclusion criteria.

Cohort features may be stored in the user group account (institution or medical service for example) and may be accessible to all the users of the same group.

### Position of a Patient Versus Cohort Based on a Single Metric

The present system may allow positioning of a patient on one selected metric against a cohort.

### Additional Information

A cohort may be constituted by:
all patients in the institution;
all patients in the clinical study cohort;
a manual cohort available in the institution;
a smart cohort available in the institution.

### Cohort Patient Outcome Visualization

The present system may be configured to generate and display comparisons of patient clinical data between different cohorts.

Clinical data may include:
PFS, OS, Response at first evaluation.

Comparative visualization may include:
Kaplan-Meyer graph, Histograms.

These outcomes and data visualization options may be state of the art in clinical study reporting.

The present system allows, given that a cohort has been created, to visualize and monitor the outcome of a cohort using the following metrics:
progression free survival, overall survival, clinical response at first evaluation

### PFS/OS Cohort Stratification

The present system may be configured to stratify a cohort in two sub-groups based on the cohort outcome (PFS/OS), selected threshold (number of months or days), and may allow access to descriptive statistical insights of the cohort stratification. The present system allows, provided that a cohort of patients has been created, to split this cohorts into two sub cohorts based on a threshold value on the PFS or OS outcome and compare the outcome and descriptive statistical analysis of these two sub-cohorts.

These comparisons may be completed by the display of the statistical significance computed for the outcome and applicable data of the descriptive statistical analysis comparison.

### Manual Cohort Management

The present system may be configured to group patients in cohorts based on user defined or selected features. In an embodiment, the system also provides the user with the ability to edit, delete, and/or duplicate cohorts.

The cohort shall be defined by at least: an identifier and a name.

Manual cohort features may be stored in the user group account (for example, institution or medical service) and may be accessible to all the users of the same group and may contain only patients belonging to the corresponding account.

A manual cohort can be shared with another account. However, such sharing may be permitted as a function of an agreement recorded from both institution and the patients of the cohort. In a further embodiment, such an agreement may manifest as a digital authentication key, wherein such a key permits extraction of data from patient records and/or permits inclusion of the patient's data in cohort processing. Further, authentication keys and/or other digital permissions may exist between institutions and/or accounts. Thus, exchange of cohorts, modification of cohorts, and/or viewing of cohorts by other accounts may be permitted as a function of digital authentication permissions. For the purposes of this disclosure, such permissions may be those known to one of ordinary skill in the art.

### Visualize Patient List Included in a Cohort

The present system may include access to the list of patients of an institution that are included in a given cohort. Accordingly, such a visualization may be generated on a client device (described in further detail below). For example, the client device may be configured to display a graphical user interface, wherein the graphical user interface comprises one or more modules adapted to display the various metrics outputted by the system. Thus, in an embodiment, the server may run predictions and the server processor may execute the actions described herein, wherein the server is further configured to deliver such resulting information to the client device. In such an embodiment, the various modules of the graphical user interface may be populated with the information originating from the server. The present system may be configured, when accessing a given cohort, to generate and display the list of patients included in the cohort.

This information may comprise:
the number of patient coming from other institution; and/or
the exhaustive list of patients coming from an institution with patient first name, last name, and ID.

### Clinical Response Cohort Stratification

The present system may be configured to stratify a cohort in two or more subgroups based on the cohort outcome (for example, Clinical Response at 1st evaluation), selected threshold (progression vs non-progression), and extract descriptive statistical insights of the cohort stratification.

The present system, provided that a cohort of patients has been created, may be configured to split this cohorts into two sub-cohorts based on a selection of categories of clinical response outcome and compare the outcome and descriptive statistical analysis of these two sub-cohorts.

These comparisons may be completed by the display of the p-values computed for the outcome and applicable data of the descriptive statistical analysis comparison.

### Patient Enrollment Status Notification

The present system may be configured to permit access to the latest patient enrollment status (number of total and new patients in user account and in the global cohorts).

### Display Patient's Cohorts

The present system may be configured to access patient cohorts (smart or manual) of which the selected patient is part of. In the case of smart cohorts, the matching may be performed in real-time, such that it is executed when the user is looking at a particular patient, given all the patient clinical data available at this date.

### Clinical Study Cohort Update Information

In an embodiment, the present system notifies the user on updates for user, user group, and clinical study cohorts, including differences in the number of patients in the cohort, identity, and characteristics of patients included or excluded from cohort since a previous update. Such updates may be delivered to a user's client device upon occurrence of such an update. For example, such updates may cause the server, client device, and/or other component of the computing system to deliver a notification to the client device. The notification may be a text alert or another alert style known to those of ordinary skill in the art. In an embodiment, the alert may inform the user of the nature of the update and/or the implications of such an update on patient monitoring or outcome predictions thereof.

### Sharing Smart Cohort Definitions across Institutions

The present system may be configured to export and import smart cohort filters. The system allows sharing of the cohort filter definitions. between users, user groups, including across institutions. This feature may permit users to build on expert knowledge and enhances the process of filter creation.

The present system, when a given smart cohort has been created, may be configured to allow export of the inclusion criteria defined for this cohort (for example, in order to share it with another institution).

### Import Smart Cohort Filter

The present system may be configured to create a cohort by importing the inclusion criteria that was shared to a user of another institution.

### Display the List of Cohorts

In an embodiment, the system may be configured to generate and display the list of cohorts that belongs to the user's account, as well as other accounts that are accessible to the user or user group.

The aforementioned list may provide information about each cohort, namely:
Cohort type,
Cohort name,
Number of patients in the cohort,
Creation date,
Last update date,
Cohort description, if available,
Bookmarked status (i.e., a user selected or curated sub-list of cohorts), and/or
If the cohort is a smart cohort: the number of criteria defining the cohort.

### Cohort Quick Search

The present system may include cohort search feature, wherein cohorts that are part of the same user group may be searched using criteria including, but not limited to:
cohort type,
cohort name,
cohort updated recently.

### Cohort Creation

The present system may be configured to allow creation of a cohort by providing:
a cohort name,
a description,
a scope (patient in the user institution or across network),
a list of inclusion criteria.

The cohorting inclusion criteria for the clinical parameters such as:
Treatment type,
Age range,
Gender,
Smoking status category,
Histopathology category,
PD-L1 expression level range,
Performance status category,
Metastases number and location,
Clinical response at treatment first evaluation,
Blood analysis measure ranges, and/or
Vital status category.

### Patient Inclusion in Cohort Notification

The present system allows, when accessing the application, to be informed of the evolution of patient inclusion in the cohorts of my account:
How many new patients from my institution have been included in the cohorts of my account;
How many new patients from other institutions have been included in the cohorts of my account.

### Cohorts Comparison

The present system, provided that the access to several cohorts is granted, may be configured to generate and/or predict comparisons of the outcome and descriptive analysis of these cohorts.

This comparison may be completed by the display of the statistical significance computed for the outcome and applicable data of the descriptive statistical analysis comparison.

### Example of Display of a Cohort

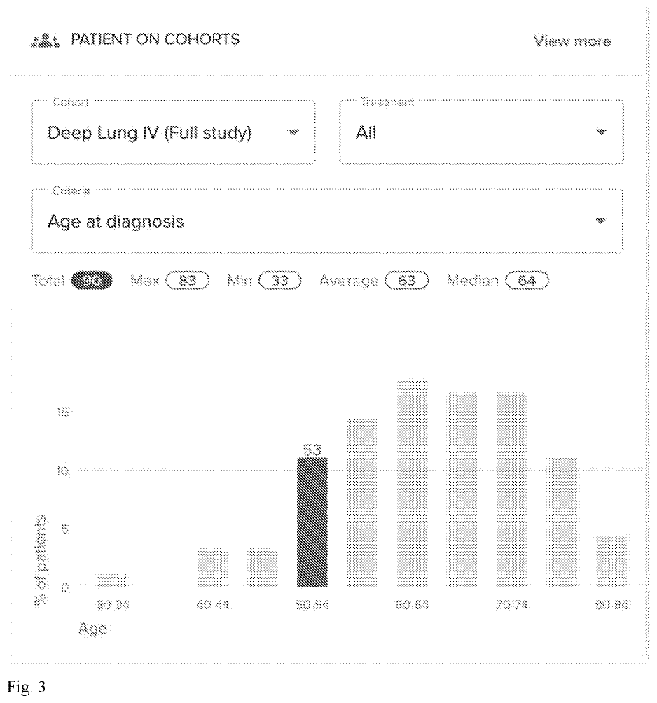

FIG. 3 shows more information about the cohort. In the chart on the bottom window, the repartition of the number of patients per age group may be displayed. For example, the groups 60-64 and 70-74 have the highest number of patients. The section 50-54 refers to the inclusion criteria of the current cohort.

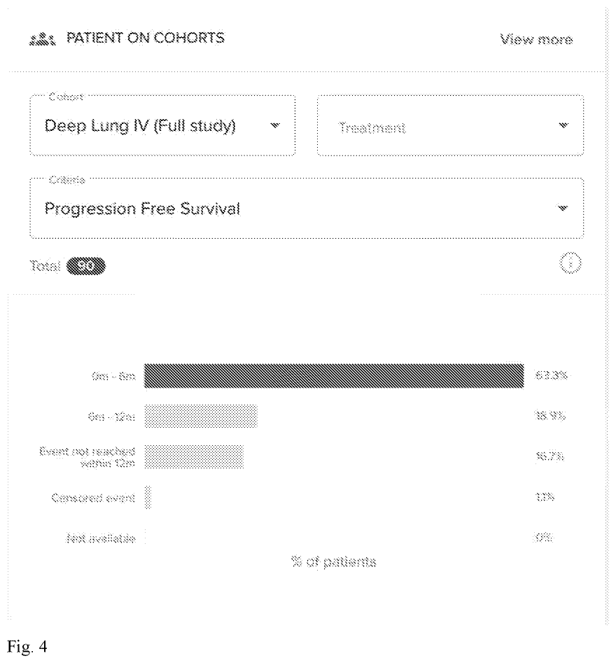

FIG. 4 displays another graph with a different set of criteria. The graph shows the PFS (Progression Free Survival) parameter expression level for the patients having received Immunotherapy only. In one embodiment of the invention, the user selects the criteria to be displayed. In one embodiment the parameter displayed is PD-L1 expression level, Performance status at first evaluation, Performance status at diagnosis, PFS, overall survival Rate (OSR), Histopathology, Metastatic status, and/or Clinical response at first evaluation.

In one embodiment, the computer system of the invention may be configured to display in more detail the distribution of the clinical criteria among a plurality of patients. The window may be split in at least 2 windows (in a preferable embodiment, at least 3 windows). The number of windows displayed may be decided by the user. The selected patients of the cohort may be identic or distinct from the patients selected for other windows.

In one embodiment, the clinical parameter may be selected among the list comprising age at diagnostic, PD-L1 Expression level, Histopathology, ECOG at diagnosis, ECOG at first evaluation, Liver metastasis at baseline, Liver Metastasis at first evaluation, brain metastasis at baseline, brain metastasis at first evaluation, clinical response at first evaluation, progression free survival or Overall survival. In one embodiment, the list of clinical parameters may also comprise radiomics features or genomics features (in particular, for example, the molecular profile such as KrasG mutational status).

In one embodiment, the computer system of the invention may be used for new hypothesis generation. By selecting various criteria among the list of clinical parameters or by selecting distinct group of patients selected by the treatment receive, the computer supports new hypothesis generation.

### Prediction

In one embodiment, the computer system is connected with a fourth database comprising a list of models for prediction of clinical outcome according to diagnostic status. The database may further comprise a list of inputting parameters for the different prediction models, the computer system may execute the additional steps of:
Selecting the model for prediction of clinical outcome according to the diagnostic status of the patient;
Retrieving the list of inputting parameters according to the model;
Inputting the data of the selected patient into the model for prediction of clinical outcome according to the diagnostic status of the patient;
Generating prediction of clinical outcome;
Displaying on an additional window the prediction of clinical outcome and confidence of the prediction.

Accordingly, as described above, the model may be a machine learning model or any other suitable predictive model.

This step of generating predictive treatment response or treatment efficacy of a patient may be based on the patient's multimodal features, wherein in one step the features may be aggregated into a vector of features' values (also referred to as feature vector values). In one embodiment, the patient's multimodal features are not complete, thus, the features are aggregated into a vector of features' values that is not complete (a non-complete vector of features' values).

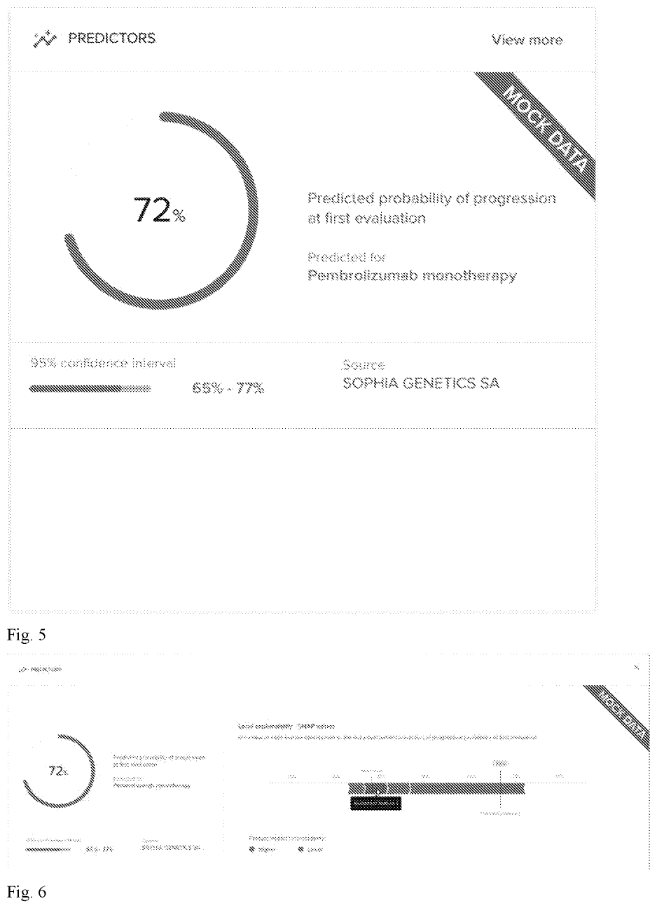

FIG. 5 displays a window wherein the prediction of clinical outcome with the confidence number is shown. As a non-limiting example, in the window, there is a 72% chance of progression at first evaluating whereby the patient is treated with Pembrolizumab monotherapy. In such a non-limiting example, in the circle beneath, a confidence interval of 95% is provided and may display the variation of the confidence number.

In FIG. 6, a further window of the prediction may be displayed, whereby the metrics of the prediction are again represented on the left side of the window. In an embodiment, on the right side of the window, an overview of the list of features contributing to the prediction is shown as a ribbon, whereby features having a lower or higher impact on the probability are displayed in a differential color (e.g. blue for a lower impact on probability and red for a higher impact on probability).

In one embodiment, it is provided a computer-implemented method of predicting treatment response or treatment efficacy of a patient based on the patient's multimodal features, wherein in one step the non-complete vector of features' values is an input to the trained imputation machine learning model and an output is a complete vector of features' values. Therefore, missing features' values are imputed by the trained imputation machine learning model.

The techniques described herein may also be implemented in electronic hardware, computer software, or any combination thereof. Such techniques may be implemented in any of a variety of devices such as general purposes computers, wireless communication device handsets, or integrated circuit devices having multiple uses including application in wireless communication device handsets and other devices. It can be executed as a stand-alone solution or in the cloud. The data storage as referenced in the above description can be a local storage, updated regularly from various sources of data, in particular other clinical personnel, or a cloud solution connecting the clinical personnel to a common platform. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general-purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules.

In general, it is understood that a “graphical user interface” (also referred to as “GUI”) refers usually to a computer program that allows users to interact with electronic devices through graphical icons and audio indicator. The GUI generally refers to a system of interactive visual components for computer software that uses windows (or display), buttons, icons, menus, pointers and scroll bars (WIMPS) interface that are selectable or clickable by the user. Example of graphical user interface are including, without being limited to: Operating System such as Mac OS, Microsoft Word, GNOME, or Internet browsers, such as Internet Explorer, Chrome, and Firefox. A “display” refers to a computer output surface and projecting mechanism that shows text and/or graphic images to the computer user in a particular area on the screen. A “window” is an area on the screen that displays information, with its contents being displayed independently from the rest of the screen. “Windows” and “Display” may thus be used interchangeably. A “menu” allows the user to execute commands by selecting from a list of choices. Options are selected with a mouse or other pointing device within a GUI. A keyboard may also be used. Menus are convenient because they show what commands are available within the computer system and/or software. A button is a graphical representation of a button that performs an action in a program when pressed.

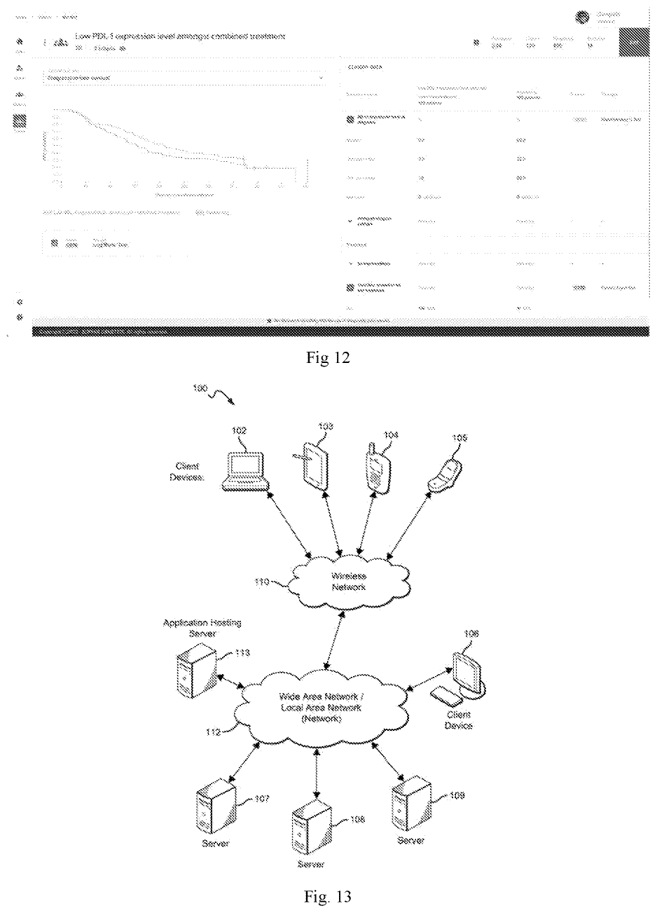

FIG. 13 illustrates components of one embodiment of an environment in which the present disclosure may be practiced. Not all of the components may be required to practice the present disclosure, and variations in the arrangement and type of the components may be made without departing from the spirit or scope of the present disclosure. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 13 also illustrates application hosting server 113.

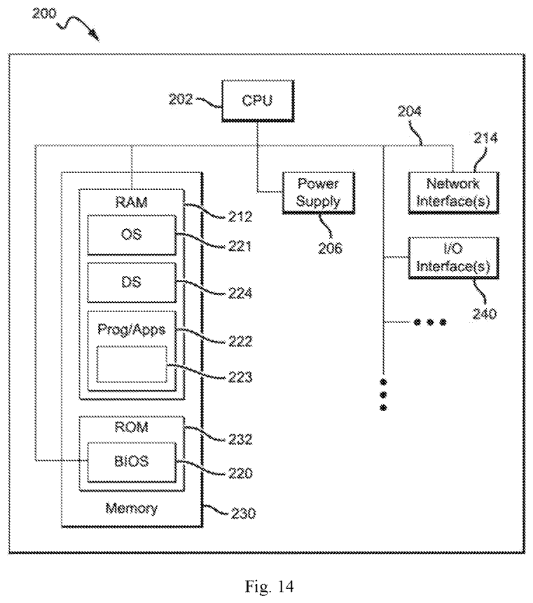

FIG. 14 illustrates a block diagram of an electronic device 200 that can implement one or more aspects of an apparatus, system and method for validating and correcting user information (the “Engine”) according to one embodiment of the present disclosure. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the present disclosure. The elements may exist on a single computer or be distributed among multiple computers, servers, devices or entities.

The power supply 206 contains one or more power components, and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www. microsoft. com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present disclosure include apparatuses, systems, and methods implementing the Engine. Embodiments of the present disclosure may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

In an embodiment, the system may receive, process, generate and/or store the clinical data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

Data originating from one or more patients may be processed via any suitable methods adapted for data capture, aggregation, processing, normalization, audio and/or visual processing, data extraction, or various other analysis purposes. Such algorithms or methods may include, but are not limited to, linguistic processing (i.e., natural language processing), statistical modeling, pattern matching, and/or trend analysis. Machine learning techniques may be used to build a communication data structure. Machine learning methods may include deep neural networks (DNNs). The DNNs may comprise recurrent neural networks (RNNs) and convolutional neural networks (CNNs). In a further embodiment, the system may include neural networks, machine learning methods, and/or artificial intelligence aspects comprising one or more feedback mechanisms. For example, the neural network, machine learning method, and/or artificial intelligence aspect may be configured to utilize the resulting output as an input for retraining via updated training data and/or subsequent repredicting via the updated neural network, updated machine learning method, and/or updated artificial intelligence aspect.

Accordingly, the system and application aspects described above may be stored, executed, processed, or otherwise performed via the hardware as described above and disclosed in FIGS. 13 and 14.

The invention of the present disclosure may be a computer system for monitoring clinical outcome, comprising a display screen and connecting at least two databases, at least one database comprising clinical data of patients, including diagnostic status, and a second database comprising a set of display parameters for each diagnostic status. In such an embodiment, the first database and the second database may be stored on one or more servers 107-109. Accordingly, a user may utilize a client device 102-106 and may be in informatic communication with the first and second database via the wireless network 110 and/or the LAN 112. In alternate embodiments, the first and second database may be stored in separate servers 107-109 (or other computing devices). For example, the first database comprising clinical data may be stored in a clinic server, while the second database comprising display characteristics for each diagnostic status may be stored in a server operated by the system administrator of the instant computer system.

In an embodiment, the computer system, for example, via the processor of a user device, may be configured to execute the steps of obtaining the clinical data for a selected patient. For example, the client device 102-106 may transmit a request for clinical data based on associations with a selected patient. In such a non-limiting example, the request may be evaluated by one or more servers 107-109.

Further, the computer system may be configured to obtain a set of display parameters based on the diagnostic status of the patient. For example, the client device 102-106 may be adapted to generate a signal and/or otherwise request the set of display parameters from the corresponding database and/or server 107-109.

In an embodiment, the databases may include separate permissions and/or authentication processes. Accordingly, the computer system may be configured to aggregate such data via electronic communication with the servers 107-109. However, in an embodiment utilizing an application hosting server 113, the client device 102-106 may communicate with the application hosting server 113, and the application hosting server 113 may more directly communicate with the servers 107-109. Thus, the application hosting server 113 may act as a bridge between the servers 107-109 and the client devices 102-106.

The processor of the client device 102-106 may be configured to display on a first window the data of the patient according to the set of display parameters. The client device 102-106 may include an image display (such as an LCD screen) configured to generate and display a GUI. The GUI may comprise at least a clinical identifier of the selected patient; a diagnostic status of the selected patient; and/or a timeline of selectable events associated with the selected patient. In such an embodiment, each selectable event may be further displayed in additional windows with the detailed metrics of the patient for the selected event. Accordingly, the computer system may include a client device in communication with one or more databases where the one or more databases are stored in one or more servers.

In an embodiment, the computer system further comprises connection to a third database. The third database may comprise at least one set of cohorting parameters, wherein the computer system may execute the additional step of selecting at least one clinical parameter of the selected patient to be compared with the corresponding clinical parameter of a cohort. In one embodiment, the clinical parameter may be selected manually by a user. In another embodiment, the clinical parameter may be determined and selected by a software element of the computer system. For example, the cohort may be formed according to a clinical parameter generated by a machine learning element or another predictive model aspect. In an embodiment where the computer system includes and/or is in communication with a client device 102-106, the client device 102-106 may transmit a request to one or more of the servers 109. The request may induce retrieval of clinical data of the plurality of patients comprised in the cohort. The system, for example via the client device or a server, may represent the distribution of the selected clinical parameter of the cohort. Accordingly, the client device processor or server processor may be configured to generate a representation of the distribution of the selected clinical parameter of the cohort. Further, either the server processor and/or client device processor may be configured to position the selected clinical parameter of the selected patient in the distribution. In a further embodiment, the client device may display in a window the distribution of the clinical parameters of the cohort.

The steps and/or aspects described herein may be executed by any of the computing devices represented in FIGS. 13 and 14.

The invention of the present disclosure may be a computer system further comprising connection to a database of trained prediction machine learning models according to the diagnostic status and a list of inputting parameters for a trained prediction machine learning model according to diagnostic status. Accordingly, the computer system may be configured to execute the additional step of selecting the trained model for prediction of clinical outcome according to the diagnostic status of the selected patient. In an embodiment, the trained model may be a machine learning model. In another embodiment, the trained model may be any category of predictive model. A client device may be configured to retrieve the list of input parameters for the patient according to the trained model. In another embodiment, a server may be configured to retrieve such a list. In an embodiment, a server may be adapted to input the data for the selected patient into the trained model for prediction of clinical outcome according to the diagnostic status of the patient. Consequently, the server may generate a prediction of clinical outcome. For example, by running the trained model and generating the prediction on the server, the client device may experience reduced processing loads. Thus, while the client device is configured to receive input from a user and output the generated results, the client device may not be bogged down by the intensive processing that is occurring externally. The client device may display, via the GUI, a fourth window showing the prediction of clinical outcome and confidence of the model.

Therefore, the computer system described herein may include one or more client devices 102-106, a plurality of servers 107-109, and an application hosting server 113. For example, the client device 102-106 may be a desktop computer or a smart device operated by the user. The client device 102-106 may be in communication with an application hosting server 113. The application hosting server 113 may be configured to execute the software aspects of the “application” as described throughout the disclosure. The application hosting server 113 may be configured to process various requests generated by the client device 102-106. The servers 107-109, for the purposes of this disclosure, may be servers corresponding to the various databases and/or sources of data. For example, each server 107-109 may correspond to one or more of: EHR records, biological data, genomic data, radiological data, clinical data, predicted data, and/or other categories of data. Further, one or more of the servers 107-109 may be operated by other users, clinics, etc. Thus, sensitive information may be exchanged between the servers 107-109 and the application hosting server 113. Such sensitive information may be utilized by the application hosting server 113 to generate predictions and/or pictorial representations of various metrics, but may not expose said sensitive information (for example, identifying portions thereof) to the client device 102-106. Therefore, the distributed computer network described above may both (1) spread intensive processing loads across various servers and computing devices; and (2) allow secure exchange of information via a dedicated application hosting server.

The invention of the present disclosure may be a system of networked devices configured to monitor patient progress and evaluate outcomes thereof. Such a system may comprise a client device comprising at least one device processor, at least one display, at least one device memory comprising computer-executable device instructions which, when executed by the at least one device processor, cause the client device to receive requests from a user and/or display results from the server. The system may further include a server in bidirectional communication with the client device, the server may comprise at least one server processor, at least one server database, at least one server memory comprising computer-executable server instructions which, when executed by the at least one server processor, cause the server to recall data specific to a particular patient, gather information pertaining to a cohort, predict outcomes of a patient and/or a cohort, and deliver such information and/or results to the client device.

However, the various actions described herein may be executed by any suitable computing device. For example, the actions, software aspects, and/or other methods described herein may solely be executed and processed via a singular device. Thus, in one embodiment, the methods and features of the computing network described herein may be executed by one of a client device, an application hosting server, and a server.

Although embodiments of the present disclosure have been described with reference to specific example embodiments, it will be evident that various modifications and changes may be made to these embodiments without departing from the broader scope of these embodiments. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived there from, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled. It is thus considered that the computer system for the monitoring of clinical outcome of the invention may be used in the form of a method for monitoring of clinical outcome. The invention is also encompassing method for generating personalized clinical decision, whereby a clinical decision is made based on the monitored clinical outcome, or the predicted clinical outcome.

Such embodiments of the inventive subject matter may be referred to herein, individually and/or collectively, by the term “invention” merely for convenience and without intending to voluntarily limit the scope of this application to any single inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A computer system for dynamically reporting to a user clinical data of patients, comprising a display interface and connected with at least two databases, at least one patient database comprising for each patient a set of clinical parameters including diagnostic status, each clinical parameter being associated with a clinical data for a given patient, and a second database comprising a set of display parameters for each diagnostic status, the computer system executing the steps of:
a. obtaining the clinical data for a selected patient from the patient database;
b. obtaining a set of display parameters based on the diagnostic status of the patient from the second database;
c. displaying on the first window the clinical data of the patient according to the set of display parameters, comprising at least:
i. clinical identifier of the selected patient;
ii. diagnostic status of the selected patient;
iii. timeline of selectable events associated with the selected patient;
wherein each selectable event can be further displayed in additional windows with the clinical data of the patient for the selected event.

2. The computer system of claim 1, wherein the selectable events are selected among: patient demographic data, diagnosis status, performance status, genomics, analysis, histopathology analysis, blood analysis results, imaging examination, treatment plan, medical history, monitoring parameter, toxicology and/or progression.

3. The computer system of claim 1, wherein the clinical patient identifier comprises patient ID, age, gender, current diagnostic status, performance status, last weight, last height and/or body mass index (BMI).

4. The computer system of claim 1, further comprising means to filter the events displayed in the timeline of selectable events.

5. The computer system of claim 1, wherein the set of display parameters comprises treatment plan, medical history, monitoring parameter and/or blood analysis results, and wherein clinical data for the selected patient are further displayed in separate windows.

6. The computer system of claim 5, wherein the medical history of the patient comprises personal history, familial history and/or risk factors.

7. The computer system of claim 1, wherein, in the case that the diagnostic status of the patient is associated with one disease, at least one clinical parameter for the selected patient is further displayed in a separate window with the variation of the clinical data of said parameter over time.

8. The computer system of claim 1, wherein the detection of a change in one patient diagnostic status entails the retrieval of a set of display parameters corresponding to the new diagnostic status and updates of the displayed clinical data for the patient in the window or windows of the system.

9. The computer system of claim 1, further comprising connection to a database of trained prediction machine learning models according to the diagnostic status and a list of inputting parameters for a trained prediction machine learning model according to diagnostic status, the computer system executing the steps of:
a. selecting the trained model for prediction of clinical outcome according to the diagnostic status of the selected patient;
b. retrieving the list of input parameters for the patient according to the trained model;
c. inputting the clinical data for the selected patient into the trained model for prediction of clinical outcome according to the diagnostic status of the patient;
d. generating prediction of clinical outcome; and
e. displaying on a fourth window the prediction of clinical outcome and confidence of the model.

10. The computer system of claim 9, wherein a list of features contributing to the prediction are further displayed in the additional window.

11. The computer system of claim 1, wherein the current diagnostic status comprises disease, stage of disease or subclassification.

12. The computer system of claim 1, wherein said system further comprises a cohort database comprising a plurality of cohorts, each cohort being represented by a set of cohorting parameters selected among the clinical parameters, each cohorting parameter having a predefined cohorting inclusion criteria said system being configured to execute the steps of:
a. selecting, from the patient database, a group of patients to study for which the clinical data match a set of study inclusion criteria of a set of study clinical parameters;
b. defining a set of cohorting inclusion criteria of a set of cohorting parameters including the set of study clinical parameters and associated inclusion criteria, said set of cohorting parameters and the inclusion criteria representing a cohort;
c. parsing the clinical data of the patient's database to retrieve the list of patients included in the cohort;
d. storing the cohorting parameters and the inclusion criteria for the cohort in the cohort database; and
e. storing in the cohort database the list of patients included in the cohort.

13. The computer system of claim 12, wherein the selecting of the group of patients include only the patients for which the clinical data of the cohorting clinical parameters is known.

14. The computer system of claim 12, further comprising at least one of steps:
a. comparing for at least one clinical parameter, the clinical data of the patients included in the cohort with the clinical data of the group of patients not included in the cohort;
b. displaying in a graphical form or via statistical report the result of the comparison;
c. determining at least one comparison metric representing a statistical significance of the difference between the clinical data of the patients within the cohort and the clinical data of the group of patients not included in the cohort; or
d. a combination thereof.

15. (canceled)

16. (canceled)

17. The computer system of claim 13, further comprising the steps of:
a. parsing the clinical data of the patient's database to update the list of patients included in the cohort; and
b. if the list of patients has changed compared to the stored list of patients, updating the comparison metric and displaying the list of new patients and the corresponding comparison metric.

18. The computer system of claim 13, wherein the computer system executes the additional steps of:
a. selecting one patient;
b. displaying and selecting at least one cohort from the cohort database for which the selected patient is part of the list of patients;
c. retrieving the clinical data of the selected patient;
d. selecting at least one clinical parameter of the selected patient to be compared with the corresponding clinical parameter of the patients included in the selected cohort;
e. retrieving the clinical data for the selected clinical parameter of the plurality of patients included in the cohort;
f. computing the distribution of the retrieved clinical data; and
g. positioning and displaying the clinical data of the selected patient in the computed distribution of the retrieved clinical data.

19. The computer system of claim 18, wherein the computer system further comprises selecting one cohort among the plurality of cohorts based on a match between the selected patient's clinical data and the cohort inclusion criteria.

20. The computer system of claim 18, wherein the system is configured to select the cohort according to the patient's diagnostic status.

21. The computer system of claim 18, wherein it is configured to execute the steps of:
a. parsing the plurality of cohorting inclusion criteria with the patient's clinical data to determine the cohorts matching the patient's clinical data; and
b. displaying and sorting the list of cohorts matching the patient's clinical data.

22. The computer system of claim 21, wherein it is configured to execute the steps of:
a. comparing the clinical data of the patients with the cohorting inclusion criteria to update the list of cohorts matching the patient's clinical data; and
b. notifying the user with the difference with the previous list of cohorts.
